(19) United States
(12) Patent Application Publication
(10) Pub. No.: US 2018/0166159 A1
(43) Pub. Date: Jun. 14, 2018

(54) AGE-ADAPTIVE PULSE OXIMETRY

(71) Applicant: OWLET BABY CARE, INC., Lehi, UT (US)

(72) Inventor: Sean Kerman, Lehi, UT (US)

(21) Appl. No.: 15/832,574

(22) Filed: Dec. 5, 2017

Related U.S. Application Data
(60) Provisional application No. 62/433,514, filed on Dec. 13, 2016.

(51) Int. Cl. G16H 10/60; A61B 5/1455; A61B 5/00; G16H 50/20

(52) U.S. Cl. CPC G16H 10/60; A61B 5/14552; A61B 5/7282; A61B 2503/08; G16H 50/20; A61B 2503/04; A61B 5/746

(57) ABSTRACT

Embodiments are directed to age-adaptive physiologic monitoring. In one scenario, a method is provided for accessing an indication of a user's age. The method next includes determining which type of physiologic monitoring is to be performed for the user, where each type of physiologic monitoring has an associated physiologic monitoring algorithm. The method also includes modifying parameters associated with the physiologic monitoring algorithm based on the indication of the user's age, and operating a physiologic monitoring system configured to provide the specified type of physiologic monitoring using the modified parameters of the physiologic monitoring algorithm.

### CROSS-REFERENCE TO RELATED APPLICATIONS

This application is claims the benefit of and priority to U.S. Provisional Patent Application Ser. No. 62/433,514 filed on Dec. 13, 2016 and entitled “AGE-ADAPTIVE PULSE OXIMETRY”. This application expressly incorporates herein the entirety of the foregoing applications.

### BACKGROUND

As people age, the physiology of their bodies naturally changes. These physiological changes can affect the operation of physiological monitoring systems. This is particularly true with infants, where physiology changes dramatically in the first year of life.

### BRIEF SUMMARY

Embodiments described herein are directed to age-adaptive physiologic monitoring. In one embodiment, a method is provided for accessing an indication of a user's age. The method next includes determining which type of physiologic monitoring is to be performed for the user, where each type of physiologic monitoring has an associated physiologic monitoring algorithm. The method also includes modifying parameters associated with the physiologic monitoring algorithm based on the indication of the user's age, and operating a physiologic monitoring system configured to provide the specified type of physiologic monitoring using the modified parameters of the physiologic monitoring algorithm.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be apparent to one of ordinary skill in the art from the description, or may be learned by the practice of the teachings herein. Features and advantages of embodiments described herein may be realized and obtained by means of the instruments and combinations particularly pointed out in the appended claims. Features of the embodiments described herein will become more fully apparent from the following description and appended claims.

### BRIEF DESCRIPTION OF THE DRAWINGS

To further clarify the above and other features of the embodiments described herein, a more particular description will be rendered by reference to the appended drawings. It is appreciated that these drawings depict only examples of the embodiments described herein and are therefore not to be considered limiting of its scope. The embodiments will be described and explained with additional specificity and detail through the use of the accompanying drawings in which:

FIG. 1 illustrates a flowchart of an example method for age-adaptive physiologic monitoring.

### DETAILED DESCRIPTION

Embodiments are generally directed to age-adaptive physiologic monitoring. In one scenario, a method for performing age-adaptive physiologic monitoring is provided. The method first includes accessing an indication of a user's age. The method next includes determining which type of physiologic monitoring is to be performed for the user, where each type of physiologic monitoring has an associated physiologic monitoring algorithm. The method also includes modifying parameters associated with the physiologic monitoring algorithm based on the indication of the user's age, and operating a physiologic monitoring system configured to provide the specified type of physiologic monitoring using the modified parameters of the physiologic monitoring algorithm.

As noted above, when people age, many changes occur to their physiology. These physiological changes can change the optimal operating conditions for physiological monitoring systems. For instance, pulse-oximiters are configured to monitor blood flow through a particular body part (e.g. a finger) and determine a heartbeat. As a patient undergoes physiological changes, the way in which a heartbeat is determined may also change. This is particularly true with infants, where physiology changes extensively in the first year of life. The embodiments disclosed herein thus describe systems and methods for changing the operating parameters of physiological monitoring systems as the age of the patient changes. Here, it should be noted that heartbeat detection is only one type of physiologic monitoring system, and that the concepts described herein may apply to many different types of physiologic monitoring systems. In each system, a patient's current age may be used to adapt the functionality of the physiological monitoring system.

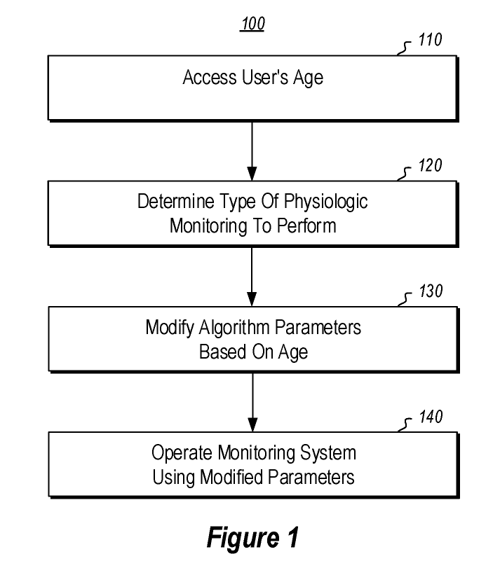

In some embodiments, the patient's age may be input by a user such as a doctor, nurse, parent or guardian or the user themselves. The system takes the patient's birth date, conception date, or other reference to patient age and implements one or more changes to the operating parameters of the physiologic monitoring system based on the patient's age. Operating parameters may include alarm thresholds, alarm delays, processing algorithms, and other operating parameters used in conjunction with the physiologic monitoring system. These concepts will be explained further below with regard to method 100 of FIG. 1.

In view of the systems and architectures described above, methodologies that may be implemented in accordance with the disclosed subject matter will be better appreciated with reference to the flow chart of FIG. 1. For purposes of simplicity of explanation, the methodologies are shown and described as a series of blocks. However, it should be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described hereinafter.

FIG. 1 illustrates a flowchart of a method 100 for age-adaptive physiologic monitoring. Method 100 includes accessing an indication of a user's age (110). The indication of the user's (i.e. the patient's) age may be provided to the physiologic monitoring system via a user interface on the system itself, or via another electronic device capable of providing a user interface. Examples of such devices may include personal computers, laptops, smart phones, wearable devices or other electronic devices. The age may include years, months, days or even minutes in the case of a newborn baby.

Method 100 next includes determining which type of physiologic monitoring is to be performed for the user, each type of physiologic monitoring having an associated physiologic monitoring algorithm (120). For example, the method may determine that heart rate monitoring is to be performed for the patient. This heart rate monitoring may be performed using a pulse-oximiter. The pulse-oximeter may be attached to a patient in a variety of different locations, but most often, it is attached to the patient's finger. The pulse-oximeter emanates light which is shined into the patient's finger. Some of this light is reflected back to an optic sensor on the pulse-oximeter. The amount of light reflected back will vary based on the amount of blood in the patient's finger. Thus, as the patient's heart beats, blood flows to and from their finger, and this rhythmic blood flow causes periodic variations in the amount of reflected light, thereby allowing a heartbeat to be detected.

Although a pulse-oximeter is described herein as an example of a physiologic monitoring system, it will be understood that substantially any type of physiologic monitoring system may be used herein. Moreover, the photoplethysmograms generated by pulse-oximiters are merely one example of a physiologic sensor signal, and it will be understood that many different types of sensor signals may be used with the systems and methods described herein.

Method 100 further includes modifying one or more parameters associated with the physiologic monitoring algorithm based on the indication of the user's age (130), and operating a physiologic monitoring system configured to provide the specified type of physiologic monitoring using the modified parameters of the physiologic monitoring algorithm (140). The parameters associated with the physiologic monitoring algorithm may include alarm thresholds, alarm delays, processing algorithms used or other types of parameters. Each of these parameters may be modified or adjusted based on the patient's age.

For instance, as mentioned above, the physiologic monitoring system may be a pulse oximetry monitoring system. When using such a system, sensor signals from the pulse oximetry monitoring system may be photoplethysmogram waveforms. These photoplethysmogram (PPG) waveforms may be fed to a Fast Fourier Transform component which converts the PPG waveforms to a frequency-domain signal, where a fundamental heart rate harmonic can be determined. This heart rate may have ranges associated with it such that, if a certain range is reached, an alarm is triggered. For instance if the heart rate is too high or too low, an alarm may be triggered which would alert a nurse or doctor to a problem.

Thus, in at least one embodiment, a parameter associated with a physiologic monitoring algorithm is an alarm threshold that, upon being reached, triggers an alarm. The alarm may be triggered immediately or after a certain duration. For example, a single occurrence of a heart rate that is too high or too low may not trigger the alarm if a delay is applied. If multiple occurrences of a heart rate that is too high or too low occur over a given time period, then the alarm may be triggered. Thus, the delay time period associated with the alarm threshold may be increased or decreased based on the current age of the user. Indeed, it will be noted that higher heart rates are common among infants and toddlers, as opposed to elderly patients who would have a lower heart rate.

Accordingly, at least one of the parameters associated with the physiologic monitoring algorithm is an alarm delay that causes triggering of an alarm to be delayed. The time period associated with the alarm delay may be increased or decreased according to the user's age or medical history. For instance, age may be used in conjunction with other factors including prior heart problems, when determining how long to delay an alarm. Other physiologic monitoring systems including respiratory monitors, brain activity monitors, blood pressure monitors or other types of monitoring systems may also have parameters thereof be modified according to the user's age.

Another such parameter associated with the physiologic monitoring algorithm is a processing algorithm. Each of the above-noted monitoring systems has one or more processing algorithms that are used, in conjunction with sensor signals, to perform the intended function. These processing algorithms may each be applied differently based on the user's age. For instance, a monitoring device may have different analysis or monitoring modes. These different modes may be activated or not activated according to the user's age. Some parameters associated with the physiologic monitoring algorithm may be changed continuously as a function of the patient's age. For instance, in the case of newborn babies, monitoring algorithms may change every few hours or minutes, as needed, to compensate for changes physiologically occurring in the baby.

The changes to the physiologic monitoring system may be implemented in discrete units. For example: a specified set of alarm criteria (e.g. criteria A) may be used for children under six months and alarm criteria B may be used for children over six months. The physiologic monitoring system may have changes applied initially and then updated at a later date, or may have changes applied continuously. For instance, an alarm delay duration may be a function of the patient's age, as follows: alarm_delay=f(patient_age), where the alarm delay is an amount of time, and the patient age is a value that may be change continuously as the patient ages. The measurements generated by the physiologic monitoring system may be displayed in a user interface, whether remote or local to the user. Display of the user interface may be triggered upon receiving the first of a series of biometric measurements from the physiologic monitoring system. The user interface may also be configured to allow a user to manually adjust the monitoring system parameters to be more or less sensitive to the patient's age.

Accordingly, methods and systems are provided for age-adaptive physiologic monitoring. These methods and systems may be used with a variety of different physiologic monitoring systems and devices.

The concepts and features described herein may be embodied in other specific forms without departing from their spirit or descriptive characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

I claim:

1. A method for age-adaptive physiologic monitoring, the method comprising:
accessing an indication of a user's age;
determining which type of physiologic monitoring is to be performed for the user, each type of physiologic monitoring having an associated physiologic monitoring algorithm;
modifying one or more parameters associated with the physiologic monitoring algorithm based on the indication of the user's age; and
operating a physiologic monitoring system configured to provide the specified type of physiologic monitoring using the modified parameters of the physiologic monitoring algorithm.

2. The method of claim 1, wherein the physiologic monitoring system comprises a pulse oximetry monitoring system.

3. The method of claim 2, wherein sensor signals from the pulse oximetry monitoring system comprise photoplethysmogram waveforms.

4. The method of claim 1, wherein at least one of the parameters associated with the physiologic monitoring algorithm comprises an alarm threshold that, upon being reached, triggers an alarm.

5. The method of claim 4, wherein a time period associated with the alarm threshold is increased or decreased according to the user's age.

6. The method of claim 1, wherein at least one of the parameters associated with the physiologic monitoring algorithm comprises an alarm delay that causes triggering of an alarm to be delayed.

7. The method of claim 6, wherein a time period associated with the alarm delay is increased or decreased according to the user's age.

8. The method of claim 1, wherein at least one of the parameters associated with the physiologic monitoring algorithm comprises a processing algorithm.

9. The method of claim 8, wherein the processing algorithm is applied differently based on the user's age.

10. The method of claim 1, wherein the parameters associated with the physiologic monitoring algorithm are continuously changed as a function of the patient's age.

11. The method of claim 1, further comprising triggering a user interface to display one or more biometric measurements of the physiologic monitoring system.
